SEQUENCE LISTING (7 sequences)

SEQ ID NO: 1 - protein, 192 aa, Homo sapiens
QCNAPEWLPFARPTNLTDEFEFPIGTYLKYECRPGYYGRPFSIICLKNSVWTGAKDRCRRKSCRNPPDPVNGMVHVIKDIQFGSQIKYSCTKGYRLIGSSSATCIISGNTVIWDNETPICDRIPCGLPPTITNGDFISTNRENFHYGSVVTYRCNPGSGGRKVFELVGEPSIYCTSNDDQVGIWSGPAPQCI

SEQ ID NO: 2 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 3 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 6 aa, synthetic construct
GGGGGG

SEQ ID NO: 5 - protein, 20 aa, synthetic construct
METDTLLLWVLLLWVPGSTG

SEQ ID NO: 6 - protein, 626 aa, synthetic construct
DGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGQCNAPEWLPFARPTNLTDEFEFPIGTYLKYECRPGYYGRPFSIICLKNSVWTGAKDRCRRKSCRNPPDPVNGMVHVIKDIQFGSQIKYSCTKGYRLIGSSSATCIISGNTVIWDNETPICDRIPCGLPPTITNGDFISTNRENFHYGSVVTYRCNPGSGGRKVFELVGEPSIYCTSNDDQVGIWSGPAPQCI

SEQ ID NO: 7 - protein, 626 aa, synthetic construct
QCNAPEWLPFARPTNLTDEFEFPIGTYLKYECRPGYYGRPFSIICLKNSVWTGAKDRCRRKSCRNPPDPVNGMVHVIKDIQFGSQIKYSCTKGYRLIGSSSATCIISGNTVIWDNETPICDRIPCGLPPTITNGDFISTNRENFHYGSVVTYRCNPGSGGRKVFELVGEPSIYCTSNDDQVGIWSGPAPQCIGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGDGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

(19) United States
(12) Patent Application Publication
(10) Pub. No.: US 2024/0391976 A1
(43) Pub. Date: Nov. 28, 2024

(54) Bi-Functional Fusion Proteins to Complement Pathways and Method of Inhibiting Bone Resorption

(71) Applicant: AP Biosciences, Inc., Taipei City (TW)

(72) Inventors: Jhong-Jhe You, Taipei City (TW); Jeng-Horng Her, San Jose, CA (US); Ren-Yeong Huang, Taipei City (TW)

(21) Appl. No.: 18/698,714

(22) PCT Filed: Nov. 10, 2022
(86) PCT No.: PCT/US22/79620

§ 371 Date: Apr. 4, 2024

Related U.S. Application Data
(60) Provisional application No. 63/281,319, filed on Nov. 19, 2021.

(51) Int. Cl. C07K 14/705; A61K 38/00; A61P 1/02

(52) U.S. Cl. CPC C07K 14/70596; A61P 1/02; A61K 38/00; C07K 2319/30

(57) ABSTRACT

The invention provides aerodynamic lift, or downward thrust for the amphibious environment, by means of two conical frames that are opposed at the bases and counter-rotating, thus cancelling out the opposing torque moment when rotating, being provided with concentric rows of fixed and retractable blades having variable angle of attack, arranged along the generatrix, separated by the fixed central area of the frame, where the power plant and the electro-mechanical elements that enable its operation are located, the cabins being situated in the conoidal hollow space of both, thus forming a body of lenticular geometry. The invention is provided with an electro-aerodynamic stabilisation system and landing gear (T), propulsion and manoeuvring being provided by pressurised air supplied through variable-area nozzles, and by the thrust due to the tilting of its axis (Y), with a fairing. Its functions and performance are similar to those of a helicopter.

### CROSS-REFERENCE TO RELATED APPLICATIONS

This application is the United States national phase of International Application No. PCT/US2022/079620, filed Nov. 10, 2022, and claims priority to U.S. Provisional Patent Application No. 63/281,319, filed Nov. 19, 2021, the disclosures of which are hereby incorporated by reference in their entireties.

The Sequence Listing associated with this application is filed in electronic format via EFS-Web and is hereby incorporated by reference into the specification in its entirety. The name of the XML file containing the Sequence Listing is NP-31022-US_SEQ_LIST.xml. The size of the XML file is 8,960 bytes, and the XML file was created on Feb. 17, 2024.

### BACKGROUND OF THE INVENTION

### Field of Invention

The present disclosure relates to a bi-functional fusion proteins and uses thereof. More particularly, the present disclosure relates to the bi-functional fusion proteins to complement pathways and uses thereof.

### Description of Related Art

Periodontitis is a highly prevalent chronic inflammatory disease which is affecting the periodontium, the tissues that surround and support the teeth. In its severe stage, periodontitis may lead to a progressive loss of the alveolar bone around the teeth and/or exert a significant systemic impact on health (Pihlstrom et al., 2005). The severe impact on the periodontium is known to be resulted from host inflammatory response to the bacteria challenge that causes periodontal tissue damage (Gaffen and Hajishengallis, 2008; Graves, 2008). The role of several pro-inflammatory cytokines, such as interleukin (IL)-1B, tumor necrosis factor-α (TNF-α), IL-6 and IL17, in the destructive periodontal inflammation is well established (Assuma et al., 1998; Cardoso et al., 2009; Dutzan et al., 2009; Gaffen and Hajishengallis, 2008; Graves, 2008; Ohyama et al., 2009; Vernal et al., 2005). A group of gram-negative anaerobic organisms, such as Porphyromonas gingivalis, Treponema denticola, and Tannerella forsythia, were known to be associated with the periodontitis (Holt and Ebersole, 2005; Socransky et al., 1998). Furthermore, those periodontitis-associated bacteria evidenced in vitro could interact with the complement system in complex ways and either inhibit or activate specific complement components (Hajishengallis et al., 2008; Krauss et al., 2010; McDowell et al., 2009; Popadiak et al., 2007; Potempa et al., 2009; Slaney et al., 2006; Wang et al., 2010). Animal models have provided mechanistic insight of how complement system could modulate or affect periodontitis (Abe et al., 2012; Hajishengallis et al., 2012; Hajishengallis et al., 2011). In supporting this, a number of clinical and histological observations also suggest involvement of components of complement, such as C3, C4, C3b, B, CD59 and C5a, etc., in pathogenesis of periodontitis (Attstrom et al., 1975; Boackle, 1991; Courts et al., 1977; Delima and Van Dyke, 2003; Ebersole, 2003; Niekrash and Patters, 1985; Patters et al., 1989; Rautemaa and Meri, 1996; Schenkein and Genco, 1977a; Schenkein and Genco, 1977b).

Complement is produced locally or systemically and plays an important role in host immune defenses. Also, it can link infection to various local or systemic inflammatory diseases, such as periodontitis (Hajishengallis, 2010; Rittirsch et al., 2008). The activation of the complement signaling cascade involves an activation cascade of signaling proteins, as well as proteolytic cleavages of serum proteins. These activation pathways of complement could be exerted via three distinct mechanisms, namely the classical, lectin (MBL), and alternative pathways (Markiewski and Lambris, 2007). All three pathways converge at the third component of complement (C3) which is known to be activated by pathway-specific C3 convertases including C4bC2a, which could form during the classical or lectin pathway of complement activation and mediate subsequent complement activation. On the other hand, upon activation by pathway-specific C3 convertase from alternative pathway, signals could also lead to generation of several effector molecules such as C3b and C5a anaphylatoxins, which play important roles in complement system. Meanwhile, Maekawa et al. had also shown that complement inhibition in nonhuman primates (NHPs) lead to inhibit inflammatory processes that result in osteoclastogenesis and eventually, bone loss. All together, these evidences suggest strongly the possibility and potential of C3-targeted intervention for treatment of human periodontitis (Maekawa et al., 2014).

Therefore, how to treating CHI3L1-induced cancer, the related art really needs to be improved.

### SUMMARY OF THE INVENTION

The present disclosure provides a bi-functional fusion protein that inhibits a complement signaling pathway, wherein the bi-functional fusion protein comprises a complement C4b binding motif, a complement C3b binding motif, and an Fc region (fragment crystallizable region).

In some embodiments, the complement C4b binding motif comprises a decay-accelerating activity (DAA) domain derived from complement receptor I.

In some embodiments, the DAA domain comprises SEQ ID NO: 1, or a sequence having at least 90% amino acid sequence identity to SEQ ID NO: 1.

In some embodiments, the complement C3b binding motif comprises a cofactor domain (CA) domain derived from complement receptor I.

In some embodiments, the CA domain comprises SEQ ID NO: 2, or a sequence having at least 90% amino acid sequence identity to SEQ ID NO: 2.

In some embodiments, the Fc region comprises an IgG domain, an IgE domain, an IgM domain, and IgD domain, an IgA domain, or an IgY domain.

In some embodiments, the IgG domain is an IgG1 domain, an IgG2 domain, an IgG3 domain, or an IgG4 domain.

In some embodiments, the IgG1 domain is SEQ ID NO: 3.

In some embodiments, the complement C4b binding motif is at N-terminal of Fc region, and the complement C3b binding motif is at C-terminal of Fc region; or the complement C3b binding motif is at N-terminal of Fc region, and the complement C4b binding motif is at C-terminal of Fc region.

In some embodiments, the bi-functional fusion protein further comprises two linkers, one of the two linkers is placed between the complement C4b binding motif and the N-terminal of Fc region, the other one of the two linkers is placed between the complement C3b binding motif and the C-terminal of Fc region; or one of the two linkers is placed between the complement C3b binding motif and the N-terminal of Fc region, the other one of the two linkers is placed between the complement C4b binding motif and the C-terminal of Fc region.

In some embodiments, each one of the two linkers is SEQ ID NO: 4.

In some embodiments, the bi-functional fusion protein is SEQ ID NO: 6 or SEQ ID NO: 7.

In some embodiments, orders of the bi-functional fusion protein comprises: the complement C4b binding motif, the complement C3b binding motif, and the Fc region; the complement C3b binding motif, the complement C4b binding motif, and the Fc region; the Fc region, the complement C4b binding motif, and the complement C3b binding motif; or the Fc region, the complement C3b binding motif, and the complement C4b binding motif.

The present disclosure also provides a pharmaceutical composition for the treatment of a complement related disease, comprising the bi-functional fusion protein as above mentioned, and at least one pharmaceutically acceptable carrier.

In some embodiments, the complement related disease comprises bone loss.

The present disclosure also provides a method of treating or preventing a complement related disease comprising administering to a patient in need thereof an effective amount of the bi-functional fusion protein as above mentioned.

In some embodiments, the complement related disease comprises bone loss characterized by a metabolic imbalance as a result of a net excess of bone resorption over bone formation.

In some embodiments, a disease associated with the bone loss is periodontal disease.

In some embodiments, the patient is human or non-human vertebrates.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 shows the structure of complement receptor I (CR1, also known as C3b/C4b receptor or CD35).

FIG. 2 shows structural design of bi-functional Fc fusion complement inhibitors.

FIG. 3 shows PAGE gel analysis of purified bi-functional complement inhibitors, SB001 and SB002, both migrate at around 130 kDa under non-reducing condition as homodimers, and as a 65 kDa monomer when reduced.

FIG. 4 and FIG. 5 show the ligand binding activity of purified SB001 and SB002 by ELISA. Ligand-coated wells are incubated with various concentrations of purified SB001 and SB002 proteins. The bound proteins are then detected with HRP-conjugated goat anti-human Fc antibody and OD₄₅₀ readings are plot.

FIG. 6 shows the inhibition of classic complement pathway using purified SB001 and SB002. Various concentrations of SB001 and SB002 diluted in GVB++ buffer are pre-incubated with GVB++ buffer-diluted human serum before addition of antibody-sensitized sheep erythrocytes. After incubation at 37° C., the reactions are stopped by centrifugation and OD₄₁₂ readings of supernatant are plotted. Inhibition of complement activation by SB001 and SB002 is determined as IC₅₀ based on inhibition of erythrocytes hemolysis.

FIG. 7 shows the inhibition of alternative pathway using purified bi-functional complement inhibitor. Various concentrations of purified bi-functional complement inhibitors in GVB⁰ buffer were pre-incubated with GVB⁰ buffer diluted human serum and then rabbit erythrocytes were applied. After incubation at 37° C., the reactions were centrifuged and the OD₄₁₂ readings of supernatant were plotted and IC₅₀ were determined to monitor the hemolytic process of erythrocytes.

FIG. 8 Immunohistochemistry (IHC) staining of C3b and C4b in human gingival tissues. Expression of complement C3b/C4b is increased in the gingival tissues of periodontal disease patients by immunohistochemistry (IHC) staining.

FIG. 9 shows the correlation between C3b/C4b expression and the severity of periodontitis progression. There are not much correlation between periodontal pocket depth and the complement C3b expression among the health, gingivitis and periodontitis groups. However, the complement C4b expression is much higher in periodontitis group than health and gingivitis group. According to these results, complement C4b may play an important role in the progression of periodontitis.

FIGS. 10A to 10E show the establishment of ligature-induced periodontitis in rat. Reconstructed three-dimensional micro-CT images for maxilla second molars. The alveolar bone level among sham-plus-vehicle (Control) and ligation-plus-vehicle (Ligation) of rats were sacrificed and subjected to micro-CT analysis on days 3, 5 and 7 (D3, D5 and D7, respectively) after ligature placement with result shown in the upper panel of FIG. 10A. Quantification analysis of alveolar bone loss was performed from reconstructed micro-CT images through the measurements of the distance between the cementoenamel junction-alveolar bone crest (CEJ-ABC) at the “furca sites”, “adjacent sites”, “around sites”, and “away sites” of the ligated molars as lower panel of FIG. 10A and FIGS. 10B to 10E.

FIGS. 11A to 11C show the protective effect of purified bi-functional proteins in ligature-induced animal model. Reconstructed three-dimensional micro-CT images for maxilla second molars. The alveolar bone level among sham-plus-vehicle (Control), ligation-plus-vehicle (Ligation), and ligation-plus-bi-functional inhibitor (15 μg/site; low dose) and 50 μg/site (high dose) (ligation+bi-functional inhibitor) groups of rats with daily administration were sacrificed (FIG. 11A) and subjected to micro-CT analysis and immunohistochemistry assay on day 7 after ligature placement with statistic result shown in the FIGS. 11B and 11C, respectively.

### DESCRIPTION OF THE INVENTION

The following disclosure provides detailed description of many different embodiments, or examples, for implementing different features of the provided subject matter. These are, of course, merely examples and are not intended to limit the disclosure but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

In some embodiments, bi-functional fusion protein provided herein have a complement C4b binding motif, a complement C3b binding motif, and a Fc region respectively including an amino acid sequence having at least about 80% identity, for example, 85%, 90%, 91%, 92%, 93%, 94%, 95%, 96%, 97%, 98%, 99%, and any number or range in between, to the amino acid sequence of SEQ ID NO: 1, SEQ ID NO: 2, and SEQ ID NO: 3.

In some embodiments, a homologue of a DAA domain which differ from a naturally occurring human DAA in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). For example, a human DAA homologue may have an amino acid sequence that is at least about 70% identical to the amino acid sequence of a naturally occurring human DAA (e.g., SEQ ID NO: 1), for example at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally occurring human DAA (e.g., SEQ ID NO: 1). In some examples, a homologue of human DAA retains all the alternative complement pathway inhibitory activity of human DAA. In some examples, the homologue of human DAA retains at least about 50%, for example, at least about any of 60%, 70%, 80%, 90%, or 95% of the complement inhibition activity of human DAA.

In some embodiments, a homologue of a CA domain which differ from a naturally occurring human CA in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). For example, a human CA homologue may have an amino acid sequence that is at least about 70% identical to the amino acid sequence of a naturally occurring human CA (e.g., SEQ ID NO: 2), for example at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally occurring human CA (e.g., SEQ ID NO: 2). In some examples, a homologue of human CA retains all the alternative complement pathway inhibitory activity of human CA. some examples, the homologue of human CA retains at least about 50%, for example, at least about any of 60%, 70%, 80%, 90%, or 95% of the complement inhibition activity of human CA.

In some embodiments, the term Fc region (fragment crystallizable region) includes at least a hinge region, a CH2 domain, and a CH3 domain. In some examples, the Fc domain is an IgG domain, an IgE domain, an IgM domain, and IgD domain, an IgA domain, or an IgY domain. Fc domains of any sequence and from any species can be used, including human, ape, monkey, mouse, rabbit, goat, and others. In some examples, Fc domains are engineered, i.e. non-naturally occurring or recombinant Fc domains generated using techniques of molecular biology, for example. In some examples, the IgG domain is an IgG1 domain, an IgG2 domain, an IgG3 domain, or an IgG4 domain.

In some embodiments, pharmaceutical composition provided herein includes any one of the bi-functional fusion protein provided herein and a pharmaceutically acceptable carrier. In some examples, the pharmaceutically acceptable carrier is conjugated to the C-terminus of one or more polypeptides of the antibody or antigen binding fragment. Any suitable means of conjugating the pharmaceutically acceptable carrier can be used, for example, including covalent conjugation and use of linkers.

In some embodiments, expression of complement components C3b and C4b in disease areas positively correlates with periodontitis pathogenic progression. We also describe the design and application of bi-functional Fc fusion proteins derived from C3b- and C4b-binding domains or motifs of human complement receptor CR1 protein which are able to inhibit activation of complement system in vitro and control or mitigate periodontitis progression in vivo. These Fc fusion proteins may be used for prevention or treatment of periodontitis-related diseases and that C3b and C4b may be used as biomarkers for pathologic progression of periodontitis.

The present disclosure describes the construction, expression and characterization of bi-functional complement inhibitors through immunoglobulin Fc region flanked by C3b and C4b binding domains or motifs, such as CA domain for C4b binding or DAA domain for C3b/C4b binding, from complement receptor 1 (CR1) directly.

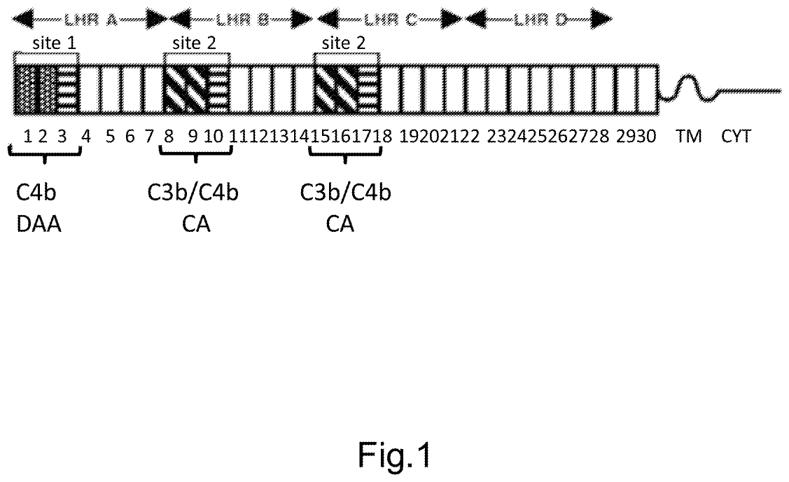

FIG. 1 shows the structure of complement receptor I (CR1, also known as C3b/C4b receptor or CD35). CR1 is a member of the regulators of complement activation family and a type I receptor composed of a 47 amino acids (a.a.) signal peptide and an extracellular domain of 1930 amino acid residues, followed by a 25-a.a. transmembrane domain (TM) and a 43-a.a. cytoplasmic region (CYT). 30 short consensus repeats (SCRs), each containing 60˜70 amino acids, are distributed in the extracellular domain of CR1. Those SCRs are further categorized as four longer regions (long homologous repeats, LHRs). Part of SCRs is also defined as domains by their functional ability, such as DAA (decay-accelerating activity) and CA (cofactor domain), which are also known as C3b or C4b associated domains, respectively.

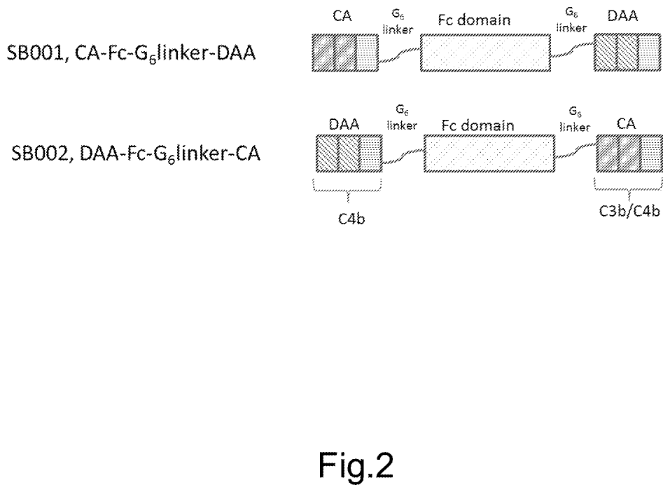

FIG. 2 shows structural design of bi-functional Fc fusion complement inhibitors. The DAA domain for C4b binding, and CA domain for C3b/C4b binding, both are derived from complement receptor type 1 (CR1), are fused to the hinge-Fc domain in two orientations, resulting in fusion proteins SB001 with orientation of CA-Fc-DAA, and SB002 with orientation of DAA-Fc-CA. The Fc domain is flanked with a short peptide linker of six glycine residues, GGGGGG (SEQ ID NO: 4), at each side to ensure correct folding of the fusion proteins.

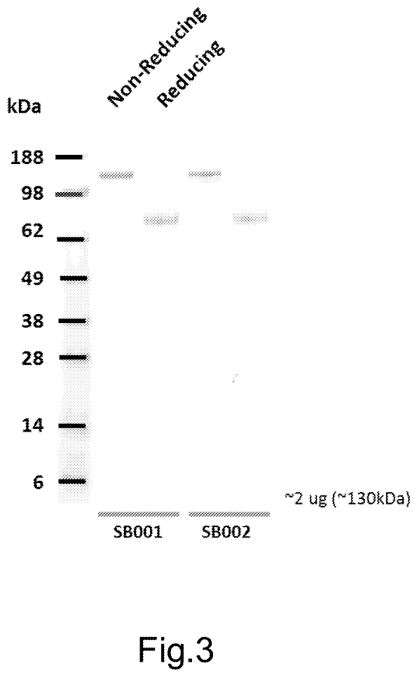

To construct bi-functional complement inhibitors by fusion of C3b and C4b binding domains or motifs (SEQ ID NO: 1 and SEQ ID NO: 2) form (CR1) through Fc region (SEQ ID NO: 3). A short flexible peptide linker, (GGGGGG) 1 (SEQ ID NO: 4) was placed between, for example, C3b/C4b binding domain (CA domain) of CR1 in N-terminal of Fc region and C4b binding domain (DAA domain) in C-terminal of Fc region to ensure correct folding and minimize steric hindrance. The coding sequences of two bi-functional complement inhibitors were shown in SEQ ID NO: 6 and NO: 7. The bi-functional Fc fusion proteins were leaded by a signal peptide (SEQ ID NO: 5) and expressed by mammalian cells, and purified from the transfected cell culture supernatant via 1-step Protein G chromatography. As shown in FIG. 3, greater than 90% purity can be obtained in a single step purification process. FIG. 3 also shows that purified fusion proteins have right molecular weight (Mw=130 kD).

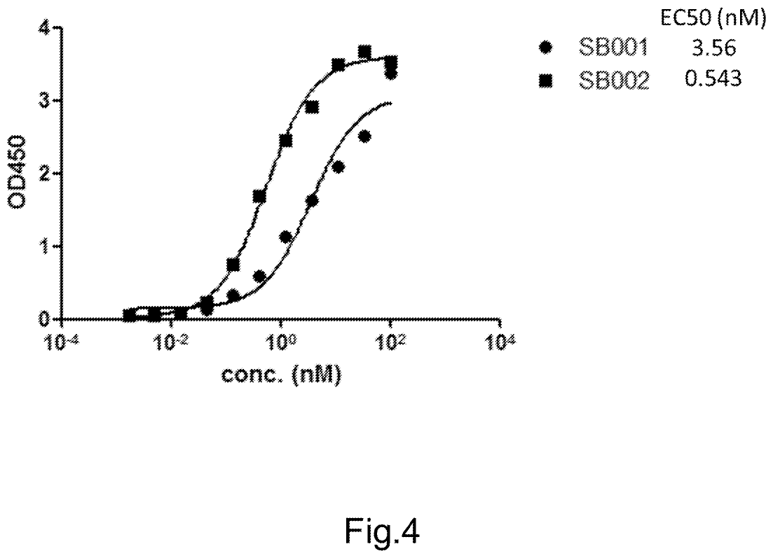

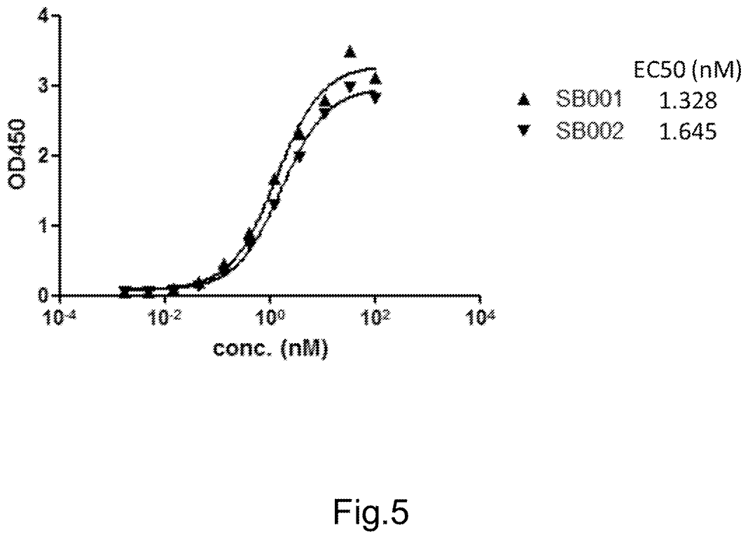

Both purified bi-functional proteins were assessed by direct C3b and C4b binding activity. Examples of binding assessment for naïve human C3b and C4b were shown in FIG. 4 and FIG. 5, respectively. Calculated EC₅₀ for both bi-functional complement inhibitors to C3b is 3.56 nM for SB001 and 0.54 nM for SB002. It revealed the SB002 possess a better C3b binding activity as comparing with SB001. However, there is no significant difference between SB001 and SB002 for C4b binding activity. The estimated EC₅₀ is 1.328 nM and 1.645 nM for SB001 and SB002, respectively.

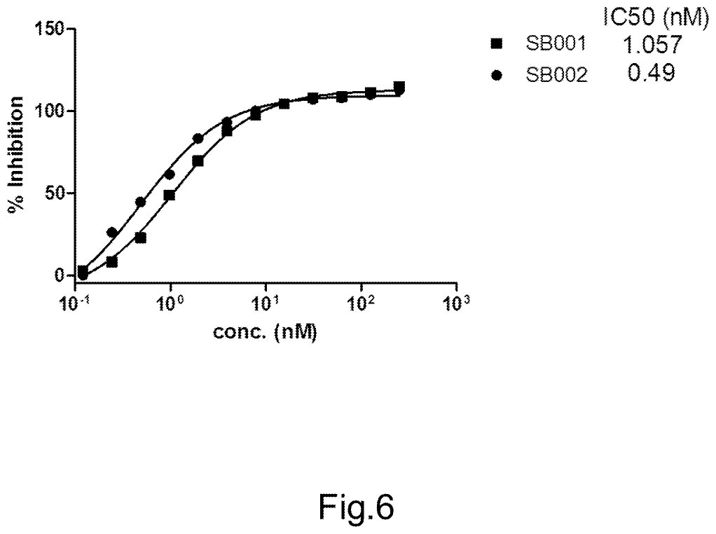

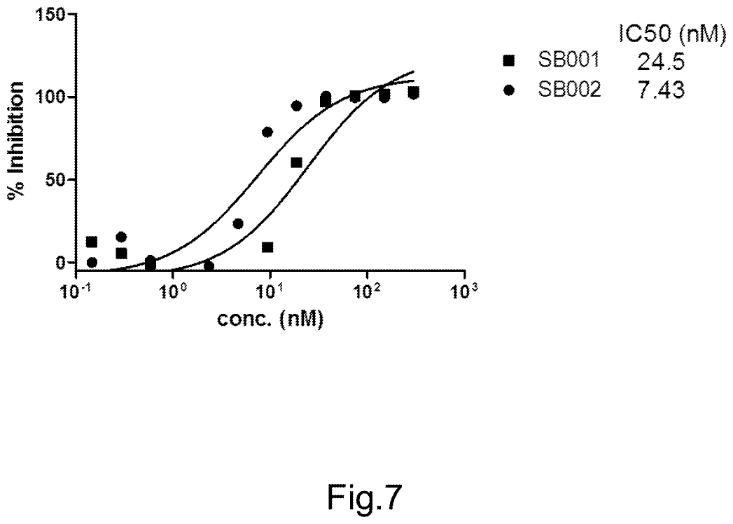

To assess inhibition performance in complement activation pathway, both bi-functional complement inhibitors were analyzed and determined for two major complement signaling pathways, classic complement pathway and alternatively complement pathway. As shown in FIG. 6, the inhibitory activity (IC₅₀) of SB001 and SB002 in classical complement pathway was 1.057 nM and 0.49 nM, respectively. It revealed the SB002 has a better inhibition performance in classical complement pathway compared with SB001. Blockage of alternative complement pathway by SB002 also exhibits a better neutralization effect in the serum (FIG. 7). The IC₅₀ for SB001 and SB002 in alternative complement pathway is 24.5 nM and 7.43 nM, respectively.

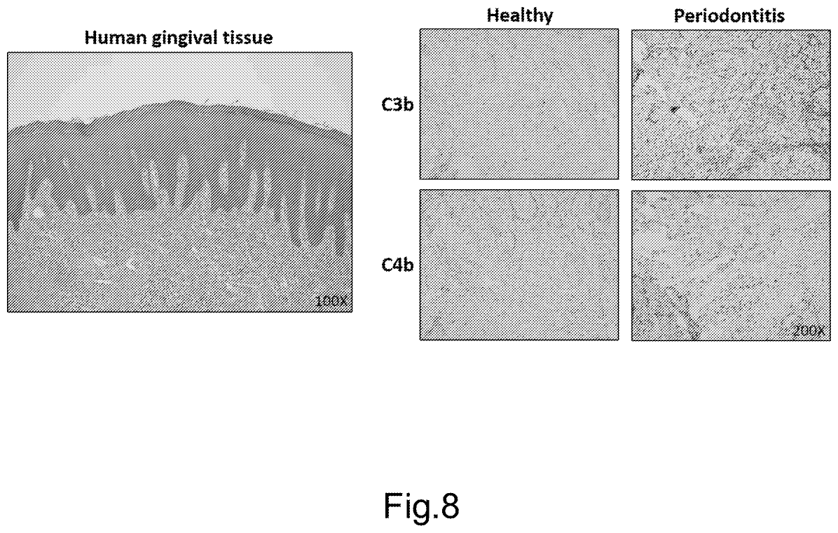

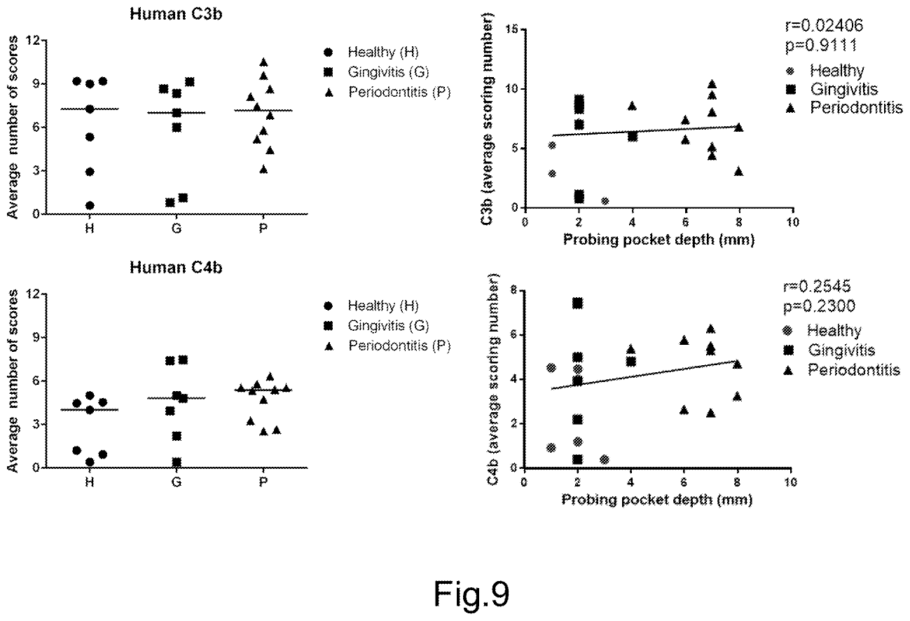

To understand the C3b/C4b expression level in the periodontal disease patient as comparing with health sample, the gingival tissue of periodontal disease patients and of health were visualized by immunohistochemistry to compare the C3b/C4b expression profile. All sample and the study protocol was approved by the local ethics committee (TSGHIRB No.2-103-05-124). As shown in the FIG. 8, either C3b or C4b expression in the gingival tissue of periodontal disease patients is significant higher than healthy. This implicate preliminarily the C3b/C4b are correlated with progression of periodontal disease. Meanwhile, the correlation between C3b/C4b expression and the severity of periodontitis progression was also investigated as revealed in the FIG. 9. The C3b expression in gingivitis or periodontitis was similar with in healthy, there is no significant correlation between periodontal pocket depth and the C3b expression among the health, gingivitis and periodontitis groups. On the contrary, C4b expression is higher than in periodontitis group as comparing with health and gingivitis group. This implicated C4b may play a major role in the progression of periodontitis.

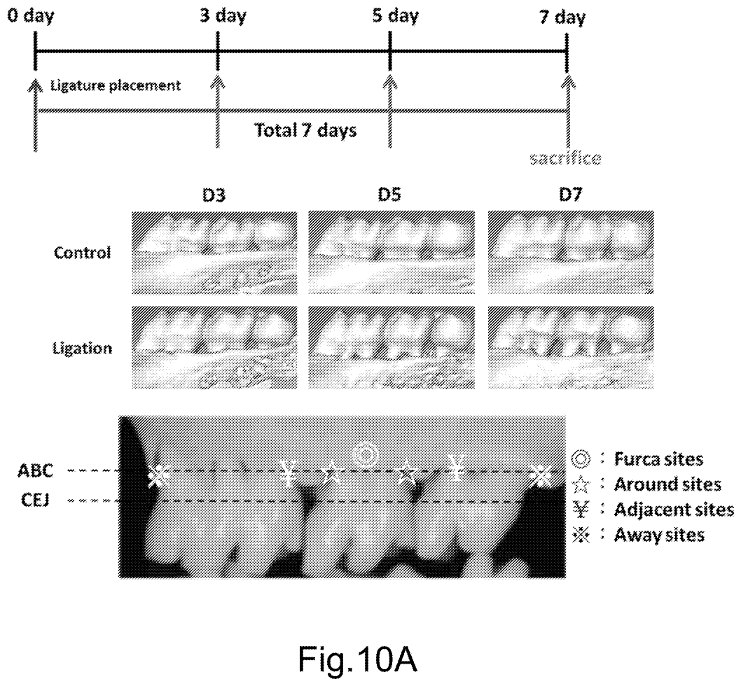

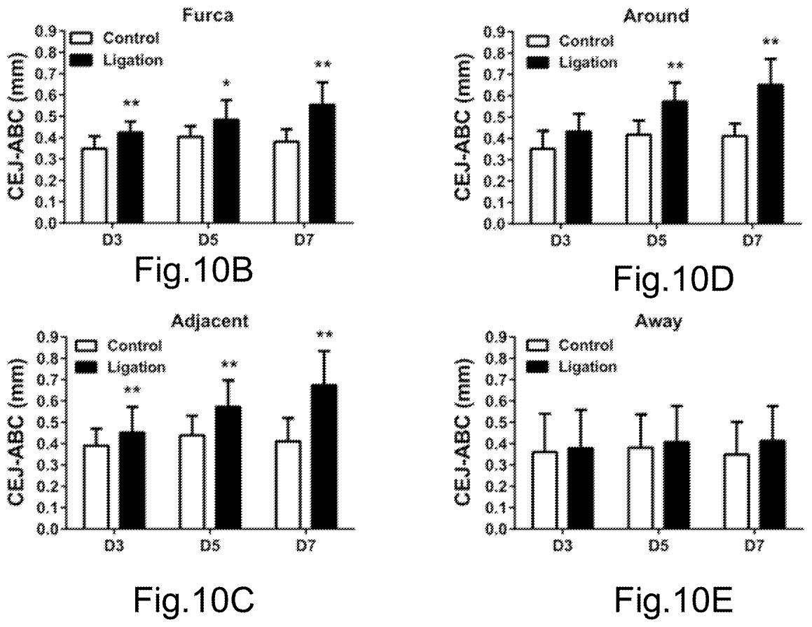

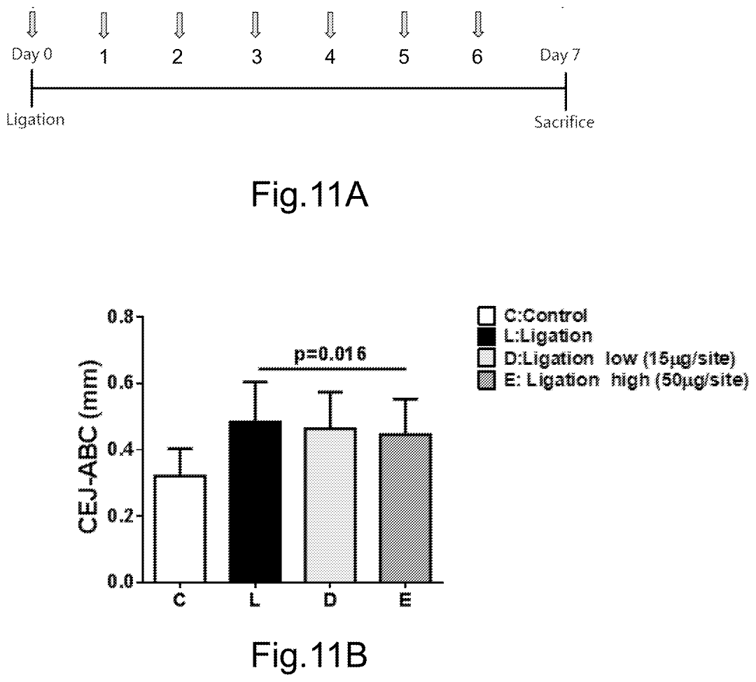

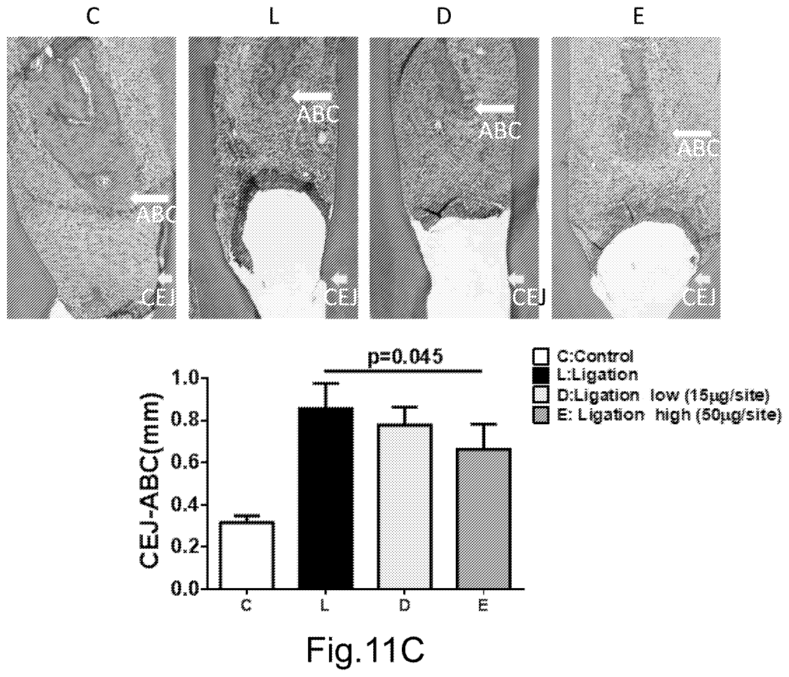

As the correlation between C3b/C4b expression and the severity of periodontitis progression was evidenced and the bi-functional complement inhibitory proteins were also showed the ability of C3b/C4b binding and trapping in vitro and in complement cell-based assay as mentioned above; therefore, to realize whether the bi-functional complement inhibitory proteins could protective or relieve the periodontitis progression is the main issue in this disclosure. Regarding for the binding affinity and inhibition performance as described above, the SB002 possess better performance as comparing with SB001; therefore, the SB002 complement inhibitor were applied for following efficiency determination assay. Here we established ligature-induced animal model to mimic the periodontitis progression in the rat. All sample and the study protocol was approved by the local ethics committee (No.IACUC-14-285). As reconstructed three-dimensional micro-CT images shown in the FIG. 10A, the alveolar bone loss was quantitated through the measurements of the distance between the cementoenamel junction-alveolar bone crest (CEJ-ABC) at the different position, such as “furca sites”, “adjacent sites”, “around sites”, and “away sites”, to represent different of periodontitis progression from severity to normal, respectively, as shown in FIGS. 10B to 10E. The result, FIGS. 11A to 11C, revealed low dose treatment with ligation (ligation+low does) was not protected or eased the disease progression after daily treatment after 7 days. However, the prevention of periodontal bone loss in high dose treatment (ligation+high does) was significant in all disease progress on day7 as comparing with low does treatment or ligation only. Collectively, these results indicated fusion proteins sustain its complement binding activity, and also able to inhibit complement signaling pathway in tested culturing condition or in ligature-induced animal model.

The human functional C3b and C4b binding domain described in the present disclosure could be any proteins or functional domains that could bind C3b/C4b such as, MCP (membrane cofactor protein), C4BP (C4 binding protein), Factor H. The Fc region in the present disclosure could be from any immunoglobulin isotypes, subclasses, allotypes, or engineered mutants, such as knob and hole Fc fragment(s).

### EXAMPLES

### Example 1 Expression and Purification of Bi-Functional Complement Pathways Inhibitory Proteins

A functional DAA and CA domains for C3b/C4b binding in human complementary receptor (CR1) was used to construct the Fc fusion proteins. The structure of bi-functional complement inhibitors, CA-Fc-DAA (SB001) and DAA-Fc-CA (SB002) were shown. The purified plasmid containing CA-Fc-DAA or DAA-Fc-CA was used to transfect 500 mL of 293 cells transiently. The culture media was harvested 96 hours post-transfection, and the Fc fusion protein was purified via Protein G chromatography. 2 g of each purified protein were PAGE gel analyzed under reducing and non-reducing conditions. As shown in FIG. 3, the molecular weight of the purified CA-Fc-DAA or DAA-Fc-CA fusion protein is about 130 kDa. Purities of the 1-step purification were >90% for both proteins.

Other C3b/C4b trapping proteins or functional domain can be generated and constructed under same schema.

### Example 2 In Vitro Binding Activity of Complement Inhibitors Against C3b and C4b

To test direct binding of purified fusion proteins to C3b or C4b on ELISA, 100 ng/well purified human C3b (CompTech, cat #A114) or C4b (CompTech, cat #A108) was coated in a 96-well ELISA plate. Various concentrations of purified complement inhibitors were then added to each well and incubated for 1 hr. After washing, 1:5000 dilution of anti-Fc HRP conjugate (Jackson Immunochemicals) was added to each well and incubated for another hour. After final washing, TMB substrate (Invitrogen Inc.) was added and OD absorbance at 450 nm was measured. The data analyzed by sigmoidal curve fitting using GraphPad Prism 5. Both fusion proteins exhibited strong binding activities against its corresponding antigen.

Other C3b/C4b trapping proteins or functional domains constructed as bi-functional fusion proteins can be generated and constructed under same schema.

### Example 3 Inhibition of Classic Complement and Alternative Pathways by Bi-Functional Inhibitor

To evaluate the complement inhibition efficiency in classic complement pathway, various concentration purified complement inhibitors (CA-Fc-DAA or DAA-Fc-CA) in GVB++ buffer (CompTech, cat #B100) were pre-incubated with diluted normal human serum for 30 minutes at 37° C. and then ˜2.5×10⁷ antibody-sensitized sheep erythrocytes (CompTech, cat #B201) were applied into the reaction for another 45 minutes, 37° C. After incubation, reactions were centrifuged and the absorbance at 412 nm of supernatant were recorded and plotted for IC₅₀ calculation.

Meanwhile, the inhibition of complement alternative pathway was also determined by similar approach. Various concentration purified complement inhibitors in GVB⁰ buffer (CompTech, cat #B103) were pre-incubated with diluted normal human serum for 30 minutes at 37° C. and then ˜2.5×10⁷ rabbit erythrocytes (CompTech, cat #B300) were applied into the reaction for another 45 minutes, 37° C. After incubation, reactions were centrifuged and the absorbance at 412 nm of supernatant were recorded and plotted for IC₅₀ determination.

Other C3b/C4b trapping proteins or functional domains constructed as bi-functional fusion proteins can be characterized under same schema.

### Example 4 Expression of C3b/C4b in Human Periodontal Biopsies

Human gingival tissue samples were harvested from selected individuals while performing periodontal surgery. The tissues were then fixed with 10% formalin and paraffin-embedded. Tissue section deparaffinized in xylene, rehydrated through serial dilutions of alcohol, immunostaining with anti-human C3b antibody or anti-human C4b antibody (Abcam). Detection by UltraVision™ Quanto Detection System (Thermo Scientific). The protocol and procedures were approved by institutional review board, Tri-Service General Hospital (2-103-05-124).

### Example 5 Correlation Between the Expression of C3b/C4b in Gum Tissues and Human Periodontitis Progression

The specimens were embedded in paraffin wax and sliced into 4-μm-thick sections. Sections were mounted on glass slides and stained with immunohistochemistry (IHC). The scoring of IHC were performed as previously described (Koo et al., 2009; Tinhofer et al., 2011). The intensity-graded of DAB (brown) in the specimens was divided into four grades (0 is unstained, 1 is weak, 2 is moderate s, 3 is strong). The DAB staining for the relative area was also divided into five grades (0 is unstained, 1 is 1˜10%, 2 is 11˜50%. 3 is 51˜80%. 4 is 81˜100%). Calculate these two scores for the IHC staining and statistically analyze the relationship between the scores and the severity of periodontitis progression.

### Example 6 Prevention of Ligature-Induced Periodontitis in Rat with Administration of Bi-Functional Complement Inhibitor

Males Sprague-Dawley rats (180-230 g) purchased from Biolasco, Inc., Taiwan. After rats were anesthetized with sodium pentobarbital (35 mg/kg, i.p.), sterile, 3-0 (diameter; 0.2 mm) black braided silk thread (surgical silk sutures; UNIK, Taipei, Taiwan) was placed around the cervix of the upper second molars bilaterally and knotted medially to induce periodontitis. The ligatures were gently displaced apically into the gingival sulci to ensure a subgingival position, and replaced when dislodged or lost. Animals were randomly allocated into two groups. The control group did not receive the induction of periodontitis and was given vehicle (normal saline) alone. The rats of ligation group were subjected to ligature placement and administrated with vehicle. Rats were sacrificed by carbon dioxide inhalation on D3, D5 and D7 after ligature (FIG. 10A).

After the final injections (Day 0), animals were sacrificed on indicated time points by carbon dioxide inhalation and maxillae specimens were dissected and fixed in 4% paraformaldehyde and prepared for micro-computerized tomography (micro-CT) and prepared for histological preparation.

For micro-CT analysis, reconstructed three-dimensional images were used to assess the distance between the cementoenamel junction (CEJ) and the coronal level of the alveolar bone crest (ABC) in all dimensions. The alveolar bone loss is defined as the measurement of the distance from CEJ to ABC (CEJ-ABC, mm).

As shown in FIGS. 10B to 10E, there was a severe bone resorption evidenced by a marked increase of CEJ-ABC distances in the ligature group when compared to the non-ligature (control) group at D5 and D7.

Next, animals were randomly allocated into four groups. The control group did not receive the induction of periodontitis and was given vehicle (normal saline) alone. The rats of ligation group were subjected to ligature placement and administrated with vehicle. Rats of ligation+bi-functional inhibitor (SB002) group were received the same ligature placement and treated daily with bi-functional inhibitor (15 μg/10 μL/site and 50 μg/10 μL/site by Micro-injection from D0 to D6) in the mucogingival tissue around maxillary 2nd molar. Rats were sacrificed by carbon dioxide inhalation on D7 after ligature.

Our results showed that there was no significant difference between ligation group (L group) and ligation low group (D group) on D7 (FIG. 11B). It is worth mention that there was a severe alveolar bone destruction evidenced by a marked increase of CEJ-ABC distances in the ligature group (L group) and ligation low group (D group) when compared to the non-ligature (control, C group) group at D7, but ligation low group (D group) has a trend to diminished alveolar bone destruction. Administration of high dose bi-functional inhibitor (E group 50 μg/site) significantly diminished alveolar bone destruction at D7 (FIG. 11C). These results clearly indicate that bi-functional inhibitor exerts a protective effect to alleviate ligature-induced periodontitis in rats.

### Example 7 Efficacy Study of Bi-Functional Complement Inhibitor in Animal Model

The histological examination and IHC staining showed that CEJ-ABC distance was markedly increased in ligated rats, which was strongly inhibited by bi-functional inhibitor (5 μg) treatment. These results clearly indicate that bi-functional inhibitor exerts a protective effect against bone resorption and decreases C3b and C4b expression in ligature-induced periodontitis.

### Example 8 Pharmacokinetic Assessment of Complement Inhibitor in Mice and Monkeys

10-40 mg/kg of bi-functional proteins, SB002 bi-functional complement inhibitory proteins will be administered into mice or monkeys via subcutaneous injection or intravenous injection. Serum samples will be taken at different time points after the injection up to 15 days. Concentrations of the Fc fusion protein in the serum samples will be determined using a sandwiched Elisa assay.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A bi-functional fusion protein that inhibits a complement signaling pathway, wherein the bi-functional fusion protein comprises a complement C4b binding motif, a complement C3b binding motif, and an Fc region.

2. The bi-functional fusion protein of claim 1, wherein the complement C4b binding motif comprises a decay-accelerating activity (DAA) domain derived from complement receptor I.

3. The bi-functional fusion protein of claim 2, wherein the DAA domain comprises SEQ ID NO: 1, or a sequence having at least 90% amino acid sequence identity to SEQ ID NO: 1.

4. The bi-functional fusion protein of claim 1, wherein the complement C3b binding motif comprises a cofactor domain (CA) domain derived from complement receptor I.

5. The bi-functional fusion protein of claim 4, wherein the CA domain comprises SEQ ID NO: 2, or a sequence having at least 90% amino acid sequence identity to SEQ ID NO: 2.

6. The bi-functional fusion protein of claim 1, wherein the Fc region comprises an IgG domain, an IgE domain, an IgM domain, and IgD domain, an IgA domain, or an IgY domain.

7. The bi-functional fusion protein of claim 6, wherein the IgG domain is an IgG1 domain, an IgG2 domain, an IgG3 domain, or an IgG4 domain.

8. The bi-functional fusion protein of claim 7, wherein the IgG1 domain is SEQ ID NO: 3.

9. The bi-functional fusion protein of claim 1, wherein
the complement C4b binding motif is at N-terminal of Fc region, and the complement C3b binding motif is at C-terminal of Fc region; or
the complement C3b binding motif is at N-terminal of Fc region, and the complement C4b binding motif is at C-terminal of Fc region.

10. The bi-functional fusion protein of claim 9, further comprising two linkers,
one of the two linkers is placed between the complement C4b binding motif and the N-terminal of Fc region, the other one of the two linkers is placed between the complement C3b binding motif and the C-terminal of Fc region; or
one of the two linkers is placed between the complement C3b binding motif and the N-terminal of Fc region, the other one of the two linkers is placed between the complement C4b binding motif and the C-terminal of Fc region.

11. The bi-functional fusion protein of claim 10, wherein each one of the two linkers is SEQ ID NO: 4.

12. The bi-functional fusion protein of claim 11, wherein the bi-functional fusion protein is SEQ ID NO: 6 or SEQ ID NO: 7.

13. The bi-functional fusion protein of claim 1, wherein orders of the bi-functional fusion protein comprises:
the complement C4b binding motif, the complement C3b binding motif, and the Fc region;
the complement C3b binding motif, the complement C4b binding motif, and the Fc region;
the Fc region, the complement C4b binding motif, and the complement C3b binding motif; or
the Fc region, the complement C3b binding motif, and the complement C4b binding motif.

14. A pharmaceutical composition for the treatment of a complement related disease, comprising the bi-functional fusion protein of claim 1, and at least one pharmaceutically acceptable carrier.

15. The pharmaceutical composition of claim 14, wherein the complement related disease comprises bone loss.

16. A method of treating or preventing a complement related disease comprising administering to a patient in need thereof an effective amount of the bi-functional fusion protein of claim 1.

17. The method of claim 16, wherein the complement related disease comprises bone loss characterized by a metabolic imbalance as a result of a net excess of bone resorption over bone formation.

18. The method of claim 17, wherein a disease associated with the bone loss is periodontal disease.

19. The method of claim 16, wherein the patient is human or non-human vertebrates.
